(19) United States
(12) Patent Application Publication
(10) Pub. No.: US 2015/0157221 A1
(43) Pub. Date: Jun. 11, 2015

(54) METHODS AND APPARATUS FOR PREDICTING HEART FAILURE EVENT

(71) Applicant: Cardiac Pacemakers, Inc., St. Paul, MN (US)

(72) Inventors: Qi An, Blaine, MN (US); Pramodsingh Hirasingh Thakur, Woodbury, MN (US); Viktoria A. Averina, Roseville, MN (US); Julie A. Thompson, Circle Pines, MN (US); Robert J. Sweeney, Woodbury, MN (US)

(21) Appl. No.: 14/548,690

(22) Filed: Nov. 20, 2014

Related U.S. Application Data
(60) Provisional application No. 61/912,588, filed on Dec. 6, 2013.

(51) Int. Cl. A61B 5/0205; A61B 5/00; A61B 5/145; A61B 7/02; A61B 5/0402; A61B 5/11

(52) U.S. Cl. CPC A61B 5/02055; A61B 5/0402; A61B 5/1118; A61B 5/1116; A61B 5/14542; A61B 7/02; A61B 5/7275; A61B 5/7282; A61B 5/021

(57) ABSTRACT

Devices and methods for detecting heart failure (HF) events or identifying patient at elevated risk of developing future HF events, such as events indicative of HF decompensation status, are described. The devices and methods can detect an HF event or predict HF risk using signal transfigurations on different portions of a physiologic signal. A system can comprise a physiologic signal analyzer circuit that can generate a signal trend of a signal feature calculated using one or more physiologic signals obtained from a patient. A signal transformation circuit can dynamically generates first and second transformations, apply the transformations to respective first and second portions of the signal trend, and generate respectively a first and second transformed signal trends. A target physiologic event detector circuit can detect a target physiologic event such as an event of worsening HF using a comparison of the first and second transformed signal trends.

### CLAIM OF PRIORITY

This application claims the benefit of priority under 35 U.S.C. §119(e) of U.S. Provisional Patent Application Ser. No. 61/912,588, filed on Dec. 6, 2013, which is herein incorporated by reference in its entirety.

### TECHNICAL FIELD

This document relates generally to medical devices, and more particularly, to systems, devices and methods for detecting and monitoring heart failure decompensation.

### BACKGROUND

Congestive heart failure (CHF) is a major health problem and affects over five million people in the United States alone. CHF is the loss of pumping power of the heart, resulting in the inability to deliver enough blood to meet the demands of peripheral tissues. CHF patients typically have enlarged heart with weakened cardiac muscles, resulting in reduced contractility and poor cardiac output of blood.

CHF is usually a chronic condition, but can occur suddenly. It can affect the left heart, right heart or both sides of the heart. If CHF affects the left ventricle, signals that control the left ventricular contraction can be delayed, and the left and right ventricles do not contract simultaneously. Non-simultaneous contractions of the left and right ventricles further decrease the pumping efficiency of the heart.

### OVERVIEW

Frequent monitoring of CHF patients and timely detection of events indicative of heart failure (HF) decompensation status can help prevent worsening of HF in CHF patients, hence reducing cost associated with HF hospitalization. Additionally, identification of patient at an elevated risk of developing future HF events such as worsening of HF can help ensure timely treatment, thereby improving the prognosis and patient outcome. Identifying and safety managing the patients having risks of future HF events can avoid unnecessary medical intervention and reduce healthcare cost.

Ambulatory medical devices can be used for monitoring HF patient and detecting HF decompensation events. Examples of such ambulatory medical devices can include implantable medical devices (MD), subcutaneous medical devices, wearable medical devices or other external medical devices. The ambulatory or implantable medical devices can include physiologic sensors which can be configured to sense electrical activity and mechanical function of the heart, or physical or physiological variables associated with the signs or symptoms associated with a new or worsening of an existing disease, such as pulmonary edema, pulmonary condition exacerbation, asthma and pneumonia, myocardial infarction, dilated cardiomyopathy, ischemic cardiomyopathy, systolic HF, diastolic HF, valvular disease, renal disease, chronic obstructive pulmonary disease, peripheral vascular disease, cerebrovascular disease, hepatic disease, diabetes, anemia, depression, pulmonary hypertension, sleep disordered breathing, or hyperlipidemia, among others.

The medical device can optionally deliver therapy such as electrical stimulation pulses to a target area, such as to restore or improve cardiac function or neural function. Some of these devices can provide diagnostic features, such as using transthoracic impedance or other sensor signals. For example, fluid accumulation in the lungs can decrease the transthoracic impedance due to the lower resistivity of the fluid than air in the lungs. Fluid accumulation in the lungs can also irritate the pulmonary system and leads to decrease in tidal volume and increase in respiratory rate. In another example, heart sounds can be useful indications of proper or improper functioning of a patient's heart. Heart sounds are associated with mechanical vibrations from activity of a patient's heart and the flow of blood through the heart. Heart sounds recur with each cardiac cycle, and according to the activity associated with the vibration, heart sounds can be separated and classified into various components including S1, S2, S3, and S4 heart sounds.

The diagnostic features obtained from the physiologic sensor signals can be used in detecting a patient's physiologic changes associated with worsening of HF status. However, because the worsening of HF can be a complex process resulting in a multitude of pathophysiologic manifestations, these diagnostic features may not always provide desired performance to timely and accurately detect or predict the worsening of HF. For example, the present inventors have recognized that the pathophysiologic manifestation of worsening of HF can be more prominent under some conditions (such as when the patient experiences elevated mental stress) than other conditions (such as when the patient is at rest). Such differences in pathophysiologic manifestation, however, may not be readily obvious from the original physiologic sensor signal, and the diagnostic features calculated using the physiologic sensor signals would not be sufficiently sensitive or specific in detecting an impending event of worsening HF. The present inventors have recognized that there remains a considerable need of methods to improve the quality and usability of the physiologic sensor signals, as well as systems and methods for using such improved physiologic sensor signals to detect events indicative or correlative of worsening of IV, or to identify CHF patients with elevated risk of developing future HF events with improved accuracy and reliability.

Various embodiments described herein can help improve detection of an HF event such as indicative of worsening of HF, or improve the process of identifying patients at elevated risk of developing future HF events. For example, a system can comprise a physiologic signal analyzer circuit that can receive one or more physiologic signals and generate a signal trend of a signal feature calculated using the physiologic signals. The system can include a signal transformation circuit that dynamically generates first and second transformations using at least one characteristic measure of the signal trend, apply the first transformation to a first portion of the signal trend to generate a first transformed signal trend, and apply the second transformation to a second portion of the signal trend different from the first portion to generate a second transformed signal trend. A target physiologic event detector circuit can detect a target physiologic event such as an event of worsening HF using a comparison of the first and second transformed signal trends.

A method can include receiving one or more physiologic signals and generating a signal trend using a signal feature calculated from the physiologic signals. The method can include dynamically generating first and second transformations using at least one characteristic measure of the signal trend, and transforming a first portion of the signal trend into a first transformed signal trend using the first transformation, and transforming a second portion of the signal trend different from the first portion into a second transformed signal trend using the second transformation. The method can also include detecting a target physiologic event in response to the transformed signal trend meeting a specified criterion.

This Overview is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates an example of a cardiac rhythm management (CRM) system and portions of the environment in which the CRM system operates.

FIG. 2 illustrates an example of a signal transformation-based HF event detection/risk assessment circuit.

FIG. 3 illustrates an example of a transformation generator.

FIG. 4 illustrates an example of a transformed signal generator for transforming first and second portions of signal trend.

FIG. 5 illustrates an example of a method for detecting a target physiologic event.

FIG. 6 illustrates an example of another method for detecting a target physiologic event.

### DETAILED DESCRIPTION

Disclosed herein are systems, devices, and methods for detecting an event indicative of worsening of HF such as an HF decompensation event, or for identifying patients with elevated risk of developing future events related to worsening of HF. The HF event detection or HF risk stratification can be performed using the physiologic signals such as sensed from one or more physiologic sensor associated with an ambulatory medical device such as an implantable cardiac device. The physiologic signals can be processed using first and second transformations based on a characteristic measure of physiologic signal trend. The first and second transformations can transform respectively specified first and second portions of the signal trend into respective first and second transformed signal trend. By analyzing the first and second transformed signal trend, the present document can provide a method and device to detect the HF event indicative of worsening of HF, or to predict the risk of future HF event, thereby allowing immediate medical attention to the patient.

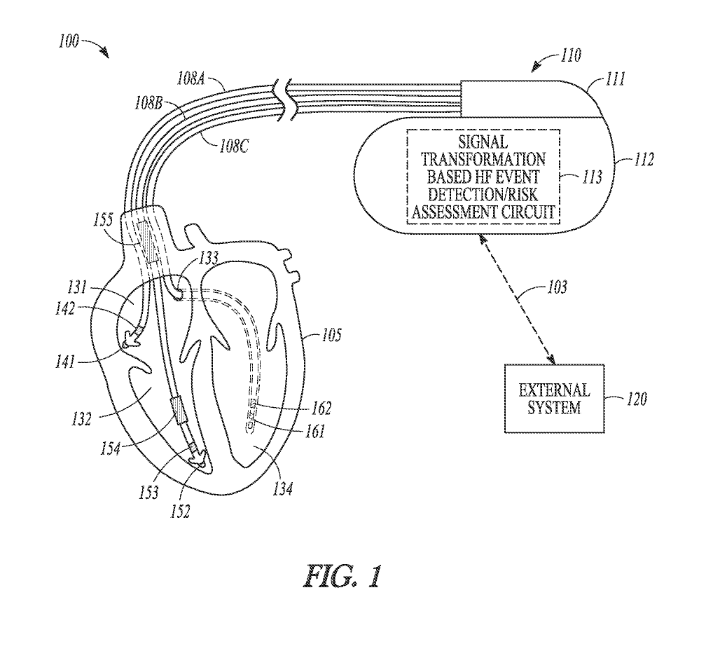

FIG. 1 illustrates an example of a Cardiac Rhythm Management (CRM) system 100 and portions of an environment in which the CRM system 100 can operate. The CRM system 100 can include an ambulatory medical device, such as an implantable medical device (IMD) 110 that can be electrically coupled to a heart 105 such as through one or more leads 108A-C, and an external system 120 that can communicate with the IMD 110 such as via a communication link 103. The IMD 110 may include an implantable cardiac device such as a pacemaker, an implantable cardioverter-defibrillator (ICD), or a cardiac resynchronization therapy defibrillator (CRT-D). The IMD 110 can include one or more monitoring or therapeutic devices such as a subcutaneously implanted device, a wearable external device, a neural stimulator, a drug delivery device, a biological therapy device, a diagnostic device, or one or more other ambulatory medical devices. The IMD 110 may be coupled to, or may be substituted by a monitoring medical device such as a bedside or other external monitor.

As illustrated in FIG. 1, the IMD 110 can include a hermetically sealed can 112 that can house an electronic circuit that can sense a physiological signal in the heart 105 and can deliver one or more therapeutic electrical pulses to a target region, such as in the heart, such as through one or more leads 108A-C. The CRM system 100 can include only one lead such as 108B, or can include two leads such as 108A and 108B.

The lead 108A can include a proximal end that can be configured to be connected to IMD 110 and a distal end that can be configured to be placed at a target location such as in the right atrium (RA) 131 of the heart 105. The lead 108A can have a first pacing-sensing electrode 141 that can be located at or near its distal end, and a second pacing-sensing electrode 142 that can be located at or near the electrode 141. The electrodes 141 and 142 can be electrically connected to the IMD 110 such as via separate conductors in the lead 108A, such as to allow for sensing of the right atrial activity and optional delivery of atrial pacing pulses. The lead 108B can be a defibrillation lead that can include a proximal end that can be connected to IMD 110 and a distal end that can be placed at a target location such as in the right ventricle (RV) 132 of heart 105. The lead 108B can have a first pacing-sensing electrode 152 that can be located at distal end, a second pacing-sensing electrode 153 that can be located near the electrode 152, a first defibrillation electrode 154 that can be located near the electrode 153, and a second defibrillation coil electrode 155 that can be located at a distance from the distal end such as for superior vena cava (SVC) placement. The electrodes 152 through 155 can be electrically connected to the IMD 110 such as via separate conductors in the lead 108B. The electrodes 152 and 153 can allow for sensing of a ventricular electrogram and can optionally allow delivery of one or more ventricular pacing pulses, and electrodes 154 and 155 can allow for delivery of one or more ventricular cardioversion/defibrillation pulses. In an example, the lead 108B can include only three electrodes 152, 154 and 155. The electrodes 152 and 154 can be used for sensing or delivery of one or more ventricular pacing pulses, and the electrodes 154 and 155 can be used for delivery of one or more ventricular cardioversion or defibrillation pulses. The lead 108C can include a proximal end that can be connected to the IMD 110 and a distal end that can be configured to be placed at a target location such as in a left ventricle (LV) 134 of the heart 105. The lead 108C may be implanted through the coronary sinus 133 and may be placed in a coronary vein over the LV such as to allow for delivery of one or more pacing pulses to the LV. The lead 108C can include an electrode 161 that can be located at a distal end of the lead 108C and another electrode 162 that can be located near the electrode 161. The lead 108C can include one or more electrodes in addition to the electrodes 161 and 162 along the body of the lead 108C. The electrodes 161 and 162, and any additional electrodes on the lead 108C, can be electrically connected to the IMD 110 such as via separate conductors in the lead 108C such as to allow for sensing of the LV electrogram and optionally allow delivery of one or more resynchronization pacing pulses from the LV.

The IMD 110 can include an electronic circuit that can sense a physiological signal. The physiological signal can include an electrogram or a signal representing mechanical function of the heart 105. The hermetically sealed can 112 may function as an electrode such as for sensing or pulse delivery. For example, an electrode from one or more of the leads 108A-C may be used together with the can 112 such as for unipolar sensing of an electrogram or for delivering one or more pacing pulses. A defibrillation electrode from the lead 108B may be used together with the can 112 such as for delivering one or more cardioversion/defibrillation pulses. In an example, the IMD 110 can sense impedance such as between electrodes located on one or more of the leads 108A-C or the can 112. The IMD 110 can be configured to inject current between a pair of electrodes, sense the resultant voltage between the same or different pair of electrodes, and determine impedance using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing. In an example, the IMD 110 can be configured to inject current between an electrode on the RV lead 108B and the can housing 112, and to sense the resultant voltage between the same electrodes or between a different electrode on the RV lead 108B and the can housing 112. A physiologic signal can be sensed from one or more physiological sensors that can be integrated within the IMD 110. The IMD 110 can also be configured to sense a physiological signal from one or more external physiologic sensors or one or more external electrodes that can be coupled to the IMD 110. Examples of the physiological signal can include one or more of heart rate, heart rate variability, electrocardiograms, intracardiac electrograms, arrhythmias, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physical activity or exertion level, physiologic response to activity, posture, respiration, body weight, or body temperature.

The arrangement and functions of these leads and electrodes are described above by way of example and not by way of limitation. Depending on the need of the patient and the capability of the implantable device, other arrangements and uses of these leads and electrodes are possible.

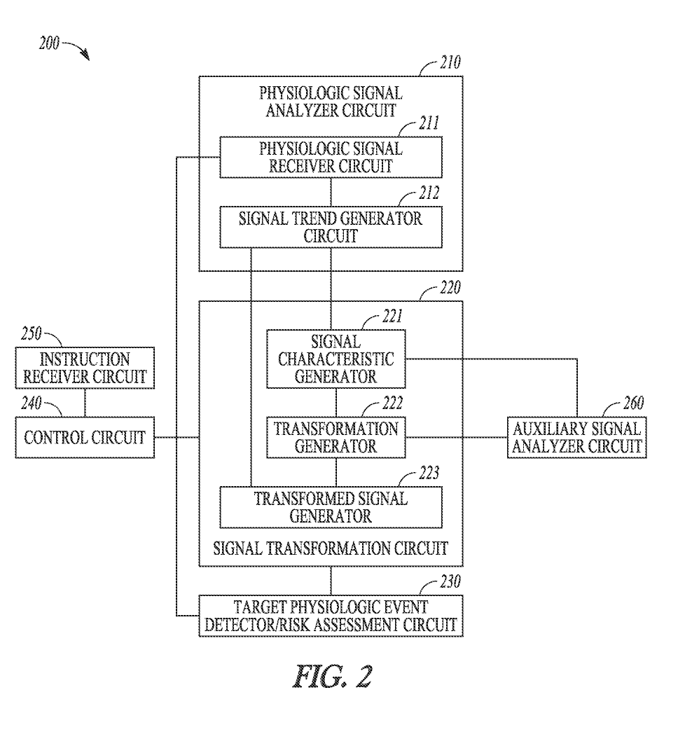

As illustrated, the CRM system 100 can include a signal transformation-based HF event detection/risk assessment circuit 113. The signal transformation-based HF event detection/risk assessment circuit 113 can receive a physiologic signal obtained from a patient and generate a trend of a signal feature using the received physiologic signal. The signal transformation-based HF event detection/risk assessment circuit 113 can generate a dynamic transformation based on characteristic of the signal trend, and transform the signal trend using the dynamic transformation. The target physiologic event detector or risk stratifier circuit can use the transformed signal trend to detect an event indicative of or correlated to worsening of HF, or to generate a composite risk indicator (CRI) indicative of the likelihood of the patient developing a future event of worsening of HF. The HF decompensation event can include one or more early precursors of an HF decompensation episode, or an event indicative of HF progression such as recovery or worsening of HF status. Examples of signal transformation-based HF event detection/risk assessment circuit 113 are described below, such as with reference to FIGS. 2-4.

The external system 120 can allow for programming of the IMD 110 and can receive information about one or more signals acquired by IMD 110, such as can be received via a communication link 103. The external system 120 can include a local external IMD programmer, or a remote patient management system that can monitor patient status or adjust one or more therapies such as from a remote location. The communication link 103 can include one or more of an inductive telemetry link, a radio-frequency telemetry link, or a telecommunication link, such as an interact connection. The communication link 103 can provide for data transmission between the IMD 110 and the external system 120. The transmitted data can include, thr example, real-time physiological data acquired by the IMD 110, physiological data acquired by and stored in the IMD 110, therapy history data or data indicating IMD operational status stored in the IMD 110, one or more programming instructions to the IMD 110 such as to configure the IMD 110 to perform one or more actions including physiological data acquisition such as using programmably specifiable sensing electrodes and configuration, device self-diagnostic test, or delivery of one or more therapies.

The signal transformation-based HF event detection/risk assessment circuit 113 may be implemented at the external system 120, which can be configured to perform HF risk stratification such as using data extracted from the IMD 110 or data stored in a memory within the external system 120. Portions of signal transformation-based HF event detection/risk assessment circuit 113 may be distributed between the IMD 110 and the external system 120.

Portions of the IMD 110 or the external system 120 can be implemented using hardware, software, or any combination of hardware and software. Portions of the IMD 110 or the external system 120 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. While described with reference to the IMD 110, the CRM system 100 could include a subcutaneous medical device (e.g., subcutaneous ICD, subcutaneous diagnostic device), wearable medical devices (e.g., patch based sensing device), or other external medical devices.

FIG. 2 illustrates an example of a signal transformation-based HF event detection/risk assessment circuit 200, which can be an embodiment of the signal transformation-based HF event detection/risk assessment circuit 113. The signal transformation-based HF event detection/risk assessment circuit 200 can also be implemented in an external system such as a patient monitor configured for presenting the patient's diagnostic information to an end-user such as a healthcare professional. The signal transformation-based HF event detection/risk assessment circuit 200 can include one or more of a physiologic signal analyzer circuit 210, a signal transformation circuit 220, a target physiologic event detector/risk assessment circuit 230, a controller circuit 240, and an instruction receiver circuit 250.

The physiologic signal analyzer circuit 210 can include a physiologic signal receiver circuit 211 and a signal trend generator circuit 212. The physiologic signal receiver circuit 211 can be configured to sense from a patient one or more physiologic signals such as using one or more physiologic sensors implanted within or attached to the patient. Examples of such a physiological signal can include one or more electrograms sensed from the electrodes on one or more of the leads 108A-C or the can 112, heart rate, heart rate variability, electrocardiogram, arrhythmia, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physiologic response to activity, apnea hypopnea index, one or more respiration signals such as a respiration rate signal or a tidal volume signal. The physiologic signals can also include one or more of brain natriuretic peptide (BNP), blood panel, sodium and potassium levels, glucose level and other biomarkers and bio-chemical markers. In some examples, the physiologic signals can be acquired from a patient and stored in a storage device such as an electronic medical record (EMR) system. The physiologic signal receiver circuit 211 can be coupled to the storage device and retrieve from the storage device one or more physiologic signals in response to a command signal. The command signal can be issued by an end-user such as via an input device coupled to the instruction receiver 250, or generated automatically by the system in response to a specified event. The physiologic signal receiver circuit 211 can include one or more sub-circuits that can perform signal conditioning or pre-processing, including signal amplification, digitization, or filtering, on the one or more physiological signals.

The signal trend generator circuit 212 can be configured to generate a plurality of signal metrics from the one or more physiologic signals, and generate a signal trend using multiple measurements of the signal metrics over a specified time period. The signal metrics can include statistical features (e.g., mean, median, standard deviation, variance, percentile, correlation, covariance, or other statistical value over a specified time segment) or morphological features (e.g., peak, trough, slope, area under the curve). The signal metrics can also include temporal information associated with the physiologic signals, such as relative timing between two physiologic events from the same or different physiologic signals. For example, the temporal information can include systolic or diastolic timing information that can be obtained from a cardiac electrical event (such as a P wave, Q wave, QRS complex, or T wave) and a cardiac mechanical event (such as a heart sound component such as S1, S2, S3 or S4 heart sounds). In an example, the signal metric can include daily maximum thoracic impedance (Z_{Max}) such as measured between electrodes located on one or more of the leads 108A-C or the can 112, and the signal trend generator circuit 212 can generate a trend of Z_{Max} by performing daily measurement of Z_{Max} over specified duration such as 3-6 months. In another example, the signal metric can include an average third (S3) heart sound strength measured during a certain time period in a day, or when an indication of patient being less active or having a specified posture is detected. The S3 strength can be trended over a sustained duration such as 0-3 months.

The signal transformation circuit 220 can be configured to transform the trend signal of the signal metric using a dynamically generated transformation. The transformation can be operated on signal amplitude, signal power, signal morphology, or signal spectral density, among others. The signal transformation circuit 220 can include a signal characteristic generator 221, a transformation generator 222, and a transformed signal generator 273.

The signal characteristic generator 221 can generate a characteristic measure using the signal trend such as produced by the signal trend generator circuit 212. In an example, the characteristic measure of the signal trend can include strength of the signal trend, such as an amplitude or peak value of an envelope of the trend signal or a rectified trend signal. In another example, the characteristic measure of the signal trend can include temporal information of the trend signal, such as relative timing of each measurement in the signal trend with respect to a reference time. The characteristic measure of the signal trend can also include mean, median, mode, standard deviation, variance, or higher-order statistical measures computed from the trend signal. Other examples of the characteristic measure of the signal trend can include difference, derivative, rate of change, or higher-order derivative or differences computed from the trend signal.

The transformation generator 222, coupled to the signal characteristic generator 221, can be configured to generate transformation Φ at least using the characteristic measure of the signal trend. The transformation Φ can be a causal transformation such that the present value (y) of the transformed signal trend can be determined using only the present or past measurements of the signal trend (x) and without using the future measurement of the signal trend, e.g., y(n)=Φ({x(k)}_{k≦n}) where n and k represent time indices. The transformation can also be non-causal transformation such that the present value of the transformed signal trend at least depends on some future measurement of the signal trend, e.g., y(n)=Φ({x(k)}) for some k>n. The transformation Φ can be a linear function such that the present value of the transformed signal trend can be a linear combination of the measurements of the signal trend. The transformation Φ can be a nonlinear function such that the present value of the transformed signal trend can include at least a nonlinear term of the measurements of the signal trend. In an example, the transformation Φ can include a plurality of weight factors. The values of the weight factors can be proportional to the strength of the signal trend.

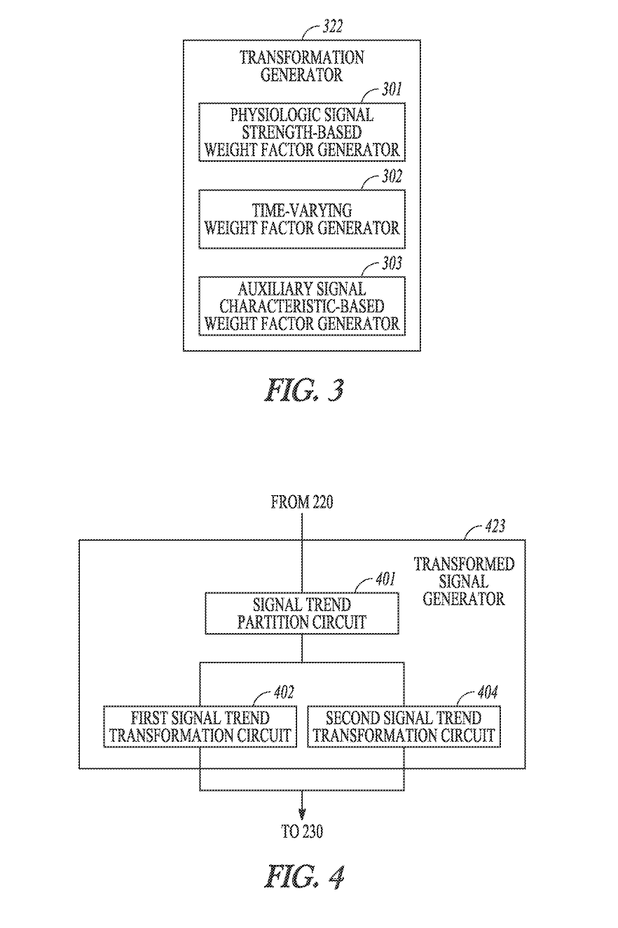

The transformation generator 222 can generate more than one transformations, and the transformed signal generator 223 can apply the transformations to specified portions of the signal trend to generate respective transformed signal trends. For example, the transformation generator 222 can generate a first transformation Φ1 and a second transformation Φ2, and the transformed signal generator 223 can apply Φ1 to a first portion of the signal trend to generate a first transformed signal trend, and apply Φ2 to a second portion of the signal trend different from the first portion of the signal trend to generate a second transformed signal trend. The first and second transformations, Φ1 and Φ2, can be different from each other. Φ1 and Φ2 can be based on the same or different characteristic measure of the signal trend. Examples of the transformation generator circuit 222 are described below, such as with reference to FIGS. 3-4.

In some examples, the signal transformation-based HF event detection/risk assessment circuit 200 can optionally include an auxiliary signal analyzer circuit 260 configured to receive an auxiliary signal. The auxillary signal can be a physiologic signal different from the one or more physiologic signals such as received by the physiologic signal receiver circuit 211. The auxiliary signal analyzer circuit 260 can be communicatively coupled to the signal characteristic generator 221 and the transformation generator 222. The signal characteristic generator 221 can generate one or more characteristic measures using the auxiliary signal in addition to or as an alternative of the signal trend produced by the physiologic signal analyzer circuit 210. Examples of the characteristic measures of the auxiliary signal can include: auxiliary signal strength such as amplitude of the auxiliary signal, or peak of the envelop or the rectified auxiliary signal; statistical measures from the auxiliary signal such as mean, median, mode, standard deviation, variance, or higher-order statistical measures computed from the auxiliary signal; morphological features extracted from the auxiliary signal; or temporal information of the auxiliary such as relative timing of each measurement in the auxiliary signal with respect to a reference time. The transformation generator 222 can dynamically generate transformation using the characteristic measures of the auxiliary signal. In an example, the transformation generator 222 can generate a plurality of weight factors proportional to the auxiliary signal strength.

The target physiologic event detector/risk assessment circuit 230 can receive the transformed signal trend from the transformed signal generator 223, and detect the presence of the target physiologic event such as an event indicative of worsening of HP when the transformed signal trend meets a specified criterion. Alternatively or additionally, the target physiologic event detector/risk assessment circuit 230 can use the transformed signal trend to predict likelihood of the patient developing a target physiologic event such as an event indicating worsening of HF, or HF decompensation in a specified timeframe, such as within approximately 1-6 months, or beyond 6 months. The target physiologic event detector/risk assessment circuit 230 can also be used to identify patients at elevated risk of developing a new or worsening of an existing disease, such as pulmonary edema, pulmonary condition exacerbation, asthma and pneumonia, myocardial infarction, dilated cardiomyopathy, ischemic cardiomyopathy, systolic HF, diastolic HF, valvular disease, renal disease, chronic obstructive pulmonary disease (COPD), peripheral vascular disease, cerebrovascular disease, hepatic disease, diabetes, anemia, depression, pulmonary hypertension, sleep disordered breathing, or hyperlipidemia, among others.

The target physiologic event detector/risk assessment circuit 230 can be configured to calculate a detection indicator (DI), and detect the target physiologic event if and when DI meets a specified criterion. In an example, the target physiologic event detector/risk assessment circuit 230 can calculate the DI as a representative value of a selected portion of the transformed signal trend such as within a time span of approximately 1-14 days. The representative value can include a mean, a median, a mode, a percentile, a quartile, or other central tendency measures of the selected portion of the transformed signal trend. The target physiologic event detector/risk assessment circuit 230 can detect the target physiologic event in response to the representative value meeting a specified criterion, such as the central tendency exceeding a specified threshold, or falling within a specified range.

In another example, the target physiologic event detector/risk assessment circuit 230 can be configured to calculate the DI using a comparison between the first and second transformed signal trends such as produced by the transformed signal generator 223. The target physiologic event detector/risk assessment circuit 230 can detect the target physiologic event if and when DI meets a specified criterion. In an example, a first and second representative values can be computed from the respective first and second transformed signal trends, and a HF event is deemed detected when a relative difference between the first and second representative values exceeds a specified threshold. The first and second representative values can each include a mean, a median, a mode, a percentile, a quartile, or other measures of central tendency of the signal metric values in the respective time window.

The target physiologic event detector/risk assessment circuit 230 can generate a report to inform, warn, or alert a system end-user when a physiologic event such as an event indicative of worsening of HF is detected, or an elevated risk of a patient developing a future HF event is indicated. The report can include a risk score with corresponding timeframe within which the risk is predicted. The report can also include recommended actions such as confirmative testing, diagnosis, or therapy options. The report can include one or more media formats including, for example, a textual or graphical message, a sound, an image, or a combination thereof. In an example, the report can be presented to the user via an interactive user interface on the instruction receiver circuit 250. The detected HF event or the risk score can also be presented to the end-user via the external device 120.

The controller circuit 240 can control the operations of the physiologic signal analyzer circuit 210, the signal transformation circuit 220, the target physiologic event detector/risk assessment circuit 230, and the data flow and instructions between these components. The controller circuit 240 can receive external programming input from the instruction receiver circuit 250 to control one or more of the receiving physiologic signals, generating signal characteristics, generating one or more transformations, transforming signal trends using the generated transformations, or performing HF event detection or risk assessment. Examples of the instructions received by instruction receiver 250 may include: selection of electrodes or sensors used for sensing physiologic signals, selection or confirmation of transformations, selection or confirmation of the auxiliary signal produced from the auxiliary signal analyzer circuit 260, or the configuration of the HF event detection. The instruction receiver circuit 250 can include a user interface configured to present programming options to the user and receive user's programming input. In an example, at least portion of the instruction receiver circuit 250, such as the user interface, can be implemented in the external system 120.

FIG. 3 illustrates an example of a transformation generator 322, which can be an embodiment of the transformation generator 222 as illustrated in FIG. 2. The transformation generator 322 can be configured to generate one or more signal transformations using signal characteristic measures calculated from one or more physiologic signals such as produced by the physiologic signal analyzer circuit 210, or one or more auxiliary signals such as produced by the auxiliary signal analyzer circuit 260. The transformations generated by the transformation generator 322 can be used by the transformed signal generator 222 to generate respective transformed signals.

The transformation generator 322 can include one or more of a physiologic signal strength-based weight factor generator 301, a time-varying weight factor generator 302, or an auxiliary signal characteristic-based weight factor generator 303. The physiologic signal strength-based weight factor generator 301 can generate a plurality of weight factors {w(n)} proportional to the signal strength of the signal trend, such as an amplitude or peak value of an envelope of the trend signal or a rectified trend signal. The weight factors can be a monotonically increasing function of the strength of the signal trend. In an example, the weight factors can be a monotonically increasing exponential function of the strength of the signal trend. Other monotonically increasing functions, include a linear, exponential, polynomial, hyperbolic, or logarithmic function, can also be used.

The time-varying weight factor generator 302 can generate a plurality of time-varying weight factors, where the values of the weight factors changes with time. In an example, the values of the time-varying weight factors can be a linear or a non-linear function of the relative time Δt of the signal trend with respect to a reference time T_{ref} such that Δt=t−T_{ref}. In another example, the values of the time-varying weight factor can be a monotonically increasing or a monotonically decreasing function of the relative time Δt. Examples of the monotonic function can include a linear, exponential, polynomial, hyperbolic, or logarithmic function, among others. The weight factors can then be used by the transformed signal generator 223 to transform the trend signal produced by the physiologic signal analyzer circuit 210.

The auxiliary signal characteristic-based weight factor generator 303 can be configured to generate a plurality of weight factors using one or more signal characteristics of an auxiliary signal such as produced by the auxiliary signal analyzer circuit 260. The auxiliary signal can be different from the one or more physiologic signals as produced by the physiologic signal analyzer circuit 210. In an example, the auxiliary signal analyzer circuit 260 can receive a thoracic impedance signal (Z) and generate a trend of daily maximum thoracic impedance signal (Z_{Max}). The auxiliary signal characteristic-based weight factor generator 303 can dynamically generate a plurality of weight factors {w(n)} proportional to the signal strength ∥Z_{Max}(n)∥ at time instant n, such that w(n)=ƒ(∥Z_{Max}(n)∥) where ƒ can be a linear or nonlinear function that preserves the relative signal strength of ∥Z_{Max}(n)∥. The physiologic signal analyzer circuit 210 can receive a heart sound (HS) signal and generate a S3 heart sound trend ∥S3∥. The transformed signal generator 223 can generate a transformed S3 heart sound trend ∥S3∥_{T} by applying the weight factors {w(n)} to ∥S3∥, such that ∥S3∥_{T}=Φ(∥S3∥)=w(n)·∥S3(n)∥=ƒ(∥Z_{Max}(n)∥)·S3(n)∥. The transformed S3 heart sound trend ∥S3∥_{T} can then be used for detecting a HF decompensation event or to predict patient's risk to experiencing a future event of worsening of HF.

The weight factors, such as those generated by the physiologic signal strength-based weight factor generator 301, the time-varying weight factor generator 302, or the auxiliary signal characteristic-based weight factor generator 303, can have the same size as the signal trend or a portion of the signal trend, such that the weight factors can be applied to the signal trend on a sample-by-sample basis. For example, if the portion of the signal trend (x) consists of N data samples x={x(1), x(2), . . . , x(n)}, then the weight factors produced by the transformation generator 322 can include N weights Φ={w(1), w(2), . . . , w(N)}, and the transformed signal generator 223 can produce the corresponding transformed signal trend (y) as y={y(1), y(2), . . . , y(N)} where for each y(i)=w(i)·x(i). In some examples, the size of the weight factors can be different from the size of the signal trend or the portions of the signal trend, and the transformation does not preserve the size of the original signal trend (x). For example, the transformation can involve a segment-by-segment weighted average of the original signal trend (x), resulting in a transformed signal trend (y) with fewer samples than the original signal trend (x).

FIG. 4 illustrates an example of a transformed signal generator 423, which can be an embodiment of the transformed signal generator 223 as illustrated in FIG. 2. The transformed signal generator 423 can be configured to transform two or more physiologic signal trends using respective transformations. The transformed signal generator 423 can include a signal trend partition circuit 401, a first signal trend transformation circuit 402, and a second signal trend transformation circuit 404.

The signal trend partition circuit 401 can be configured to generate at least a first portion (X1) and a second portion (X2) of the signal trend such as produced by the physiologic signal analyzer circuit 210. X1 and X2 can be taken from signal trends generated from the same or different physiologic signals. If taken from the same signal trend, X1 can be different from X2 such that X1 includes at least data from the signal trend not shared with X2. X1 can be overlapped or non-overlapped with X2. In an example, X2 can include data from the signal trend preceding X1 in time, X2 can be taken from a second time window longer than the first time window from which X1 is taken, and at least a portion of the second time window precedes the first time window in time, and X2 represents a baseline free of predicted target physiologic event. In an example, X1 and X2 are two segments of S3 strength (∥S3∥) trend signal, where X2 can represent a baseline ∥S3∥ trend free of predicted target physiologic event. As an example, X2 can be approximately 1-3 month before the first portion of the signal trend. The window size for X2 can be approximately 5-60 days, and the window size for X1 can be approximately 1-14 days.

The first signal trend transformation circuit 402 can apply a first transformation (Φ1) to the first portion of the signal trend (X1) to generate a first transformed signal trend (X1_{T}), such that X1_{T}=Φ1(X1). Likewise, the second signal trend transformation circuit 404 can apply a second transformation (Φ2) to the second portion of the signal trend (X2) to generate a second transformed signal trend (X2_{T}), such that X2_{T}=Φ2(X2). The transformations Φ1 and Φ2, which can be generated by the transformation generator 222, can be based on different characteristic measures calculated from the physiologic signal such as produced by the physiologic signal analyzer circuit 210. In an example, Φ1 can include first plurality of weight factors {w1(n)} proportionally to the amplitude of X1, that is, w1(n)=ƒ(∥X1(n)∥), where ƒ can be a linear or nonlinear function that preserves the relative signal strength of X1; and Φ2 can include a second plurality of weight factors {w2(n)} proportionally to the relative time (Δt_{X2}) of X2 with respect to a reference time that is, w2(n)=g(Δt_{X2}(n)), where g can be a linear or nonlinear, or a monotone increasing or monotone decreasing function, such as an exponential, a polynomial, a hyperbolic, or a logarithmic function, among others. The first signal trend transformation circuit 402 and the second signal trend transformation circuit 404 can generate transformed signal trends respectively as shown in Equations (1) and (2):

X1_{T}(n)=Φ1(X1(n))=w1(n)·X1(n)=ƒ(X1(n)∥)·X1(n) (1)

X2_{T}(n)=Φ2(X2(n))=w2(n)·X2(n)=g(Δt_{X2}(n))·X2(n) (2)

In another example, Φ1 can include a first plurality of time-varying weight factors {w1(n)} as a monotonically increasing function g₁ of relative time (Δt_{X1}) of X1 with respect to a first reference time, that is, w1(n)=g₁(Δt_{X1}(n)); and Φ2 can include a second plurality of time-varying weight factors {w2(n)} as a monotonically decreasing function g₂ of relative time (Δt_{X2}) of X2 with respect to a second reference time, that is, w2(n)=g₂(Δt_{X2}(n)). g₁ and g₂ can each be a linear, or nonlinear function such as an exponential, a polynomial, a hyperbolic, or a logarithmic function, among others. The first signal trend transformation circuit 402 and the second signal trend transformation circuit 404 can generate transformed signal trends respectively as shown in Equations (3) and (4):

X1_{T}(n)=(X1(n))=w1(n)·X1(n)=g₁(Δt_{X1}(n))·X1(n) (3)

X2_{T}(n)=Φ2(X2(n))=(n)·X2(n)=g₂(Δt_{X2}(n))·X2(n) (4)

In some examples, one or both of the transformations Φ1 and Φ2 can be determined using characteristic measures calculated from the auxiliary signal (U) such as produced the auxiliary signal analyzer circuit 260. The plurality of weight factors {w1(n)} and {w2(n)} can then be determined as specified functions of the signal characteristics of the respective portions of the auxiliary signal. Under the conditions corresponding to Equations (1)-(4), the first signal trend transformation circuit 402 and the second signal trend transformation circuit 404 can respectively generate transformed signal trends X1_{T} and X2_{T} using the Equations (1′)-(4′):

X1_{T}(n)=Φ1(X1(n))=w1(n)·X1(n)=ƒ(∥U1(n)∥)·X1(n) (1′)

X2_{T}(n)=Φ2(X2(n))=w2(n)·X2(n)=g(Δt_{U2}(n))·X2(n) (2′)

X1_{T}(n)=Φ1(X1(n))=w1(n)·X1(n)=g₁(Δt_{U1}(n))·X1(n) (3′)

X2_{T}(n)=Φ2(X2(n))=w2(n)·X2(n)=g₂(Δt_{U2}(n))·X2(n) (4′)

FIG. 5 illustrates an example of a method 500 for detecting a target physiologic event such as indicative of worsening of HF. The method 500 can be implemented and operate in an ambulatory medical device or in a remote patient management system. In an example, the method 500 can be performed by the signal transformation-based HF event detection/risk assessment circuit 113 implemented in the BID 110, or the external device 120 which can be in communication with the IMD 110.

At 501, one or more physiologic signal can be received from a patient. Examples of such a physiological signal can include one or more electrograms sensed from the electrodes on one or more of the leads 108A-C or the can 112, heart rate, heart rate variability, electrocardiogram, arrhythmia, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physiologic response to activity, apnea hypopnea index, one or more respiration signals such as a respiration rate signal or a tidal volume signal. The physiologic signals can also include one or more of brain natriuretic peptide (BNP), blood panel, sodium and potassium levels, glucose level and other biomarkers and bio-chemical markers. The physiologic signals can be sensed using one or more physiologic sensors associated with the patient, or be acquired from a patient and stored in a storage device such as an electronic medical record (EMR) system.

The received physiologic signals can then be processed, including signal amplification, digitization, resampling, filtering, or other signal conditioning processes. One or more signal metrics can be calculated from the one or more physiologic signals, and a signal trend can be generated at 502 using multiple measurements of the signal metrics over a specified time period. The signal metrics can include statistical features (e.g., mean, median, standard deviation, variance, percentile, correlation, covariance, or other statistical value over a specified time segment), morphological features (e.g., peak, trough, slope, area under the curve), or temporal features including relative tinning between two physiologic events from the same or different physiologic signals (e.g., systolic or diastolic timing obtained using an electrocardiogram or intracardiac electrogram and a heart sound signal). The trend of the signal metric can be generated continuously as new physiologic signal is acquired. The trend can also be generated when patient physiologic responses, ambient environment parameters, or other contextual parameters meeting specified conditions. For example, the trend can be generated only when patient is awake, the activity level is within specified range, the heart rate falls within a specified range or pacing or other device therapy are present or absent.

At 503, one or more transformations can be generated using at least one characteristic measure of the signal trend. The characteristic measure of the signal trend can include strength of the signal trend or temporal information of the trend signal. The strength of the signal trend can include an amplitude or peak value of the envelope of the trend signal or the rectified trend signal. The temporal information of the trend signal can include relative timing of each measurement in the signal trend with respect to a reference time. The characteristic measure of the signal trend can also include mean, median, mode, standard deviation, variance, or higher-order statistical measures computed from the trend signal. Other examples of the characteristic measure of the signal trend can include difference, derivative, rate of change, or higher-order derivative or differences computed from the trend signal.

The transformation can be a causal transform such that the present value of the transformed signal trend can be determined using only the present or past measurements of the signal trend. The transformation can be a non-causal transformation such that the present value of the transformed signal trend at least depends on some future measurement of the signal trend. The transformation can be linear such that the present value of the transformed signal trend can be linear combination of the measurements of the signal trend. The transformation can be non-linear such that the present value of the transformed signal trend can include at least a nonlinear term on the measurements of the signal trend.

The transformation can include a plurality of weight factors proportional to the signal strength of the signal trend. The transformation can include a plurality of time-varying weight factors. The weight factors can be calculated using linear or a non-linear functions of the relative time Δt of the signal trend with respect to a reference time T_{ref} such that Δt=t−T_{ref}. In some examples, the time-varying weight factors can be calculated using monotonically increasing or monotonically decreasing functions of the relative time Δt. Examples of the monotonic function can include a linear, exponential, polynomial, hyperbolic, or logarithmic function, among others.

In an example where first and second transformations are generated at 503, the first and second transformations can be based on different characteristic measures of the physiologic signal. For example, the first transformation can include a first plurality of weight factors proportionally to the strength of S3 heart sound ∥S3∥, while the second transformation can include a second plurality of weight factors, different from the first plurality of weight factors, that are proportionally to the relative time the ∥S3∥ trend with respect to a reference time.

The first and second transformations can be of different functions. For example, the first transformations can include a first plurality of time-varying weight factors as a monotonically increasing function of relative time of ∥S3∥ trend with respect to a first reference time, while the second transformation can include a second plurality of time-varying weight factors as a monotonically decreasing function of relative time of ∥S3∥ with respect to a second reference time.

At 504, the first and second transformations can be applied respectively to first (X1) and second (X2) portions of the signal trend to generate first and second transformed signal trends. X1 and X2 can be taken from signal trends generated from the same or different physiologic signals. If taken from the same signal trend, X1 can be different from X2 such that X1 includes at least data from the signal trend not shared with X2. X1 can be overlapped or non-overlapped with X1 in an example, X2 can be taken from a second time window longer than the first time window from which X1 is taken, and at least a portion of the second time window precedes the first time window in time. X2 can a baseline signal trend free of predicted target physiologic event. As an example, X2 can be approximately 1-3 month before the first portion of the signal trend. The window size for X2 can be approximately 5-60 days, and the window size for X1 can be approximately 1-14 days.

In an example where the first and second transformation includes respective plurality of weight factors, the size of the weight factors can be the same as the size of the respective portions of the signal trend, such that the weight factors can be applied to the signal trend on a sample-by-sample basis. For example, if the portion of the signal trend (x) consists of N data samples x={x(1), x2, . . . , x(n)}, then the weight factors generated at 503 can include N weights Φ={w(1), w(2), . . . , w(N)\, and the transformed signal generator 223 can produce the corresponding transformed signal trend (y) as y=y(1), y(2), . . . , y(N)} where for each y(i)=w(i)·x(i). In some examples, the size of the weight factors can be different from the size of the signal trend or the portions of the signal trend, and the transformation does not preserve the size of the original signal trend (x). For example, the transformation can involve a segment-by-segment weighted average of the original signal trend (x), resulting in a transformed signal trend (y) with fewer samples than the original signal trend (x).

At 505, a physiologic event such as indicative of worsening of HF can be detected using the transformed signal trends. A detection indicator (DI) can be calculated using a comparison between the first and second transformed signal trends, and to detect the target physiologic event in response to the DI meeting a specified criterion. In an example, a first and second representative values can each be computed respectively from the first and second transformed signal trends, and a HF event is deemed detected when the relative difference between the first and second representative values exceeds a specified threshold. The first and second representative values can each include a mean, a median, a mode, a percentile, a quartile, or other measures of central tendency of the signal metric values in the respective time window,

A risk index can be generated at 505 and reported to an end-user. A report can be generated to inform, warn, or alert an end-user when a physiologic event such as an event indicative of worsening of HF is detected, or an elevated risk of a patient developing a future HF event is indicated. The report can include a risk score with corresponding timeframe within which the risk is predicted. The report can also include recommended actions such as confirmative testing, diagnosis, or therapy options. The report can include one or more media formats including, for example, a textual or graphical message, a sound, an image, or a combination thereof. The risk index thus calculated can also be used to identify patients at elevated risk of developing a new or worsening of an existing disease, such as pulmonary edema, pulmonary condition exacerbation, asthma and pneumonia, myocardial infarction, dilated cardiomyopathy, ischemic cardiomyopathy, systolic HP, diastolic HF, valvular disease, renal disease, chronic obstructive pulmonary disease (COPD), peripheral vascular disease, cerebrovascular disease, hepatic disease, diabetes, anemia, depression, pulmonary hypertension, sleep disordered breathing, or hyperlipidemia, among others.

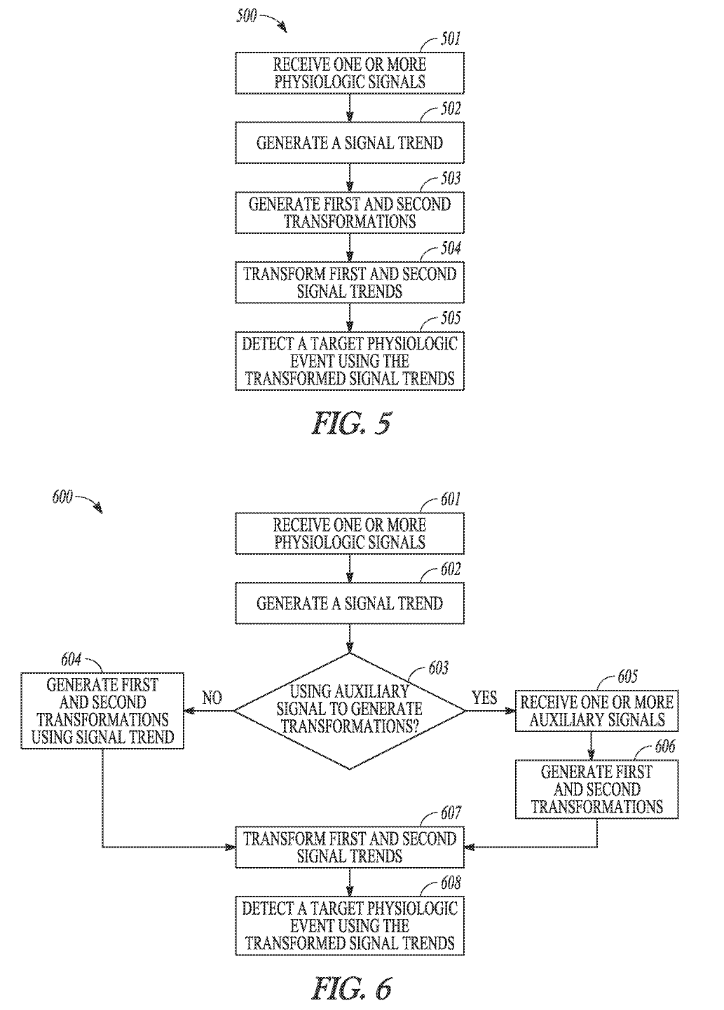

FIG. 6 illustrates an example of a method 600 for detecting a target physiologic event such as indicative of worsening of HF. The method 600 can be implemented and operate in an ambulatory medical device or in a remote patient management system. In an example, the method 600 can be performed by the signal transformation-based HF event detection/risk assessment circuit 113 implemented in the IMD 110, or the external device 120 which can be in communication with the IMD 110.

At 601, one or more physiologic signal can be received from a patient. One or more signal metrics, such as statistical, morphological, or temporal features of the signal, can be calculated from the one or more physiologic signals. At 602, a signal trend can be generated using multiple measurements of the signal metrics over a specified time period. At 603, a decision is made as to whether an auxiliary signal is to be used to generate transformation. The decision can be made based on the detected or empirical information of the physiologic signals received at 601, including one or more of signal quality, signal-to-noise ratio, signal reliability in consideration of the electrode position, lead integrity, sufficiency of the signal trend data for determining the transformation. The decision can also be made in reference to the empirical information obtained from patient historical physiologic data, which is suggestive of usability or reliability of the physiologic signal in determining the transformation.

If an auxiliary signal is not selected, then at 604, one or more transformations can be generated using at least one characteristic measure of the signal trend. If an auxiliary signal is selected, then one or more auxiliary signals can be received at 605. The auxiliary signal can be a physiologic signal different from the one or more physiologic signals received at 601. The auxiliary signal can also include non-physiologic signals such as ambient environmental signals. Characteristic measures can be calculated from the auxiliary signal, including auxiliary signal strength such as amplitude of the auxiliary signal, peak of the envelop or the rectified auxiliary signal; statistical measures from the auxiliary signal such as mean, median, mode, standard deviation, variance, or higher-order statistical measures computed from the auxiliary signal; morphological features extracted from the auxiliary signal; or temporal information of the auxiliary signal, such as relative timing of each measurement in the auxiliary signal with respect to a reference time.

At 606, one or more transformations, such as first and second transformations, can be generated using the auxiliary signals. The first and second transformations can be causal or non-causal transformations, or linear or nonlinear transformations. In an example, the first and second transformations can be of the same type of transformation (such as weight factors) yet based on different characteristic measures of the auxiliary signal. For example, the first transformation can include a first plurality of weight factors proportionally to the strength of an auxiliary signal trend, while the second transformation can include a second plurality of weight factors, different from the first plurality of weight factors, that are proportionally to the relative time the auxiliary signal trend with respect to a reference time. The first and second transformations can be of different functions. For example, the first transformations can include a first plurality of time-varying weight factors as a monotonically increasing function of relative time of the auxiliary signal trend with respect to a first reference time, while the second transformation can include a second plurality of time-varying weight factors as a monotonically decreasing function of relative time of auxiliary signal trend with respect to a second reference time. Examples of the monotonic function can include a linear, an exponential, a polynomial, a hyperbolic, or a logarithmic function, among others,

At 607, a transformed first and second signal trends can be generated. The first and second transformations, such as generated at 604, or at 605 can be applied respectively to the first (X1) and second (X2) portions of the signal trend to generate first and second transformed signal trends. X1 and X2 can be taken from the same trend signal at different time. X2 can include data from the signal trend preceding X1 in time. For example, X2 can be taken from a second time window longer than the first time window from which X1 is taken, and at least a portion of the second time window precedes the first time window in time. X2 can a baseline signal trend free of predicted target physiologic event. As an example, X2 can be approximately 1-3 month before the first portion of the signal trend. The window size for X2 can be approximately 5-60 days, and the window size for X1 can be approximately 1-14 days. In an example when the transformation include a plurality of weight factors {w(n)}, the transformed signal trend (X_{T}) can be determined by applying the weight factors {w(n)} sample-by-sample to the signal trend X generated at 602, such that X_{T}(n)=w(n)·X(n).

At 608, a physiologic event such as indicative of worsening of HF can be detected using the transformed signal trends. A detection indicator (DI) can be calculated using a comparison between the first and second transformed signal trends, and to detect the target physiologic event in response to the DI meeting a specified criterion, such as when the relative difference between the first and second representative values exceeds a specified threshold. A report can also be generated to inform, warn, or alert an end-user when a physiologic event such as an event indicative of worsening of HF is detected, or an elevated risk of a patient developing a future HF event is indicated. The report can include a risk score with corresponding timeframe within which the risk is predicted. The report can also include recommended actions such as confirmative testing, diagnosis, or therapy options.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the fill scope of equivalents to which such claims are entitled.

What is claimed is:

1. A system, comprising:
a physiologic signal analyzer circuit, including:
a physiologic signal receiver circuit configured to receive one or more physiologic signals; and
a signal trend generator configured to calculate a signal feature from the one or more physiologic signals and to generate a signal trend of the signal feature;
a signal transformation circuit configured to dynamically generate first and second transformations using at least one characteristic measure of the signal trend, apply the first transformation to a first portion of the signal trend to generate a first transformed signal trend, and apply the second transformation to a second portion of the signal trend to generate a second transformed signal trend, the second portion of the signal trend different from the first portion of the signal trend; and
a target physiologic event detector circuit configured to detect a target physiologic event using a comparison of the first and second transformed signal trends.

2. The system of claim 1, wherein the first portion of the signal trend does not overlap in time with the second portion of the signal trend.

3. The system of claim 1, wherein:
the second portion of the signal trend includes data from the signal trend preceding the first portion of the signal trend in time, the second portion of the signal trend representing a baseline free of predicted target physiologic event; and
the target physiologic event detector circuit is configured to detect the target physiologic event using a relative difference between the first and second transformed signal trends.

4. The system of claim 1, wherein the signal transformation circuit is configured to:
generate the at least one characteristic measure including strength of the signal trend; and
generate the first and second transformations each including a plurality of weight factors proportional to the strength of the signal trend.

5. The system of claim 1, wherein the signal transformation circuit is configured to generate the first and second transformations each including a plurality of time varying weight factors, the first transformation being different from the second transformation.

6. The system of claim 5, wherein the signal transformation circuit is configured to determine values of the plurality of time-varying weight factors as a linear or a non-linear function of relative time of the signal trend with respect to a reference time.

7. The system of claim 5, wherein the signal transformation circuit is configured to determine values of the plurality of time-varying weight factors as a monotonically increasing or monotonically decreasing function of relative time of the signal trend with respect to a reference time.

8. The system of claim 5, wherein the signal transformation circuit is configured to determine values of the plurality of time-varying weight factors as an exponential function of relative time of the signal trend with respect to a reference time.

9. The system of claim 5, wherein the first transformation includes a first plurality of time-varying weight factors and the second transformation includes a second plurality of time-varying weight factors, and wherein the signal transformation circuit is configured to:
determine values of the first plurality of time-varying weight factors as a monotonically increasing function of relative time of the first portion of the signal trend with respect to a first reference time; and
determine values of the second plurality of time-varying weight factors as a monotonically decreasing function of relative time of the second portion of the signal trend with respect to a second reference time.

10. The system of claim 1, comprising an auxiliary signal analyzer circuit configured to receive an auxiliary signal non-identical to the one or more physiologic signals, wherein the signal transformation circuit is configured to:
generate the at least one characteristic measure including auxiliary signal strength; and
dynamically generate the first and second transformations including a plurality of weight factors proportional to the auxiliary signal strength.

11. A system, comprising:
a physiologic signal analyzer circuit, including:
a physiologic signal receiver circuit configured to receive one or more physiologic signals; and
a signal trend generator configured to calculate a signal feature from the one or more physiologic signals and to generate a signal trend of the signal feature;
a signal transformation circuit configured to dynamically generate a transformation using strength of the signal trend, apply the transformation to the signal trend to generate a transformed signal trend using the transformation; and
a target physiologic event detector circuit configured to calculate a representative value using the transformed signal trend, and to detect a target physiologic event in response to the representative value meeting a specified criterion.

12. The system of claim 11, wherein:
the signal transformation circuit is configured to generate the transformation including a plurality of weight factors proportional to the strength of the signal trend; and
the target physiologic event detector is configured to calculate the representative value including a central tendency of a selected portion of the transformed signal trend, and to detect the target physiologic event in response to the central tendency falling within a specified range.

13. A method, comprising:
receiving one or more physiologic signals;
generating a signal trend using a signal feature calculated from the one or more physiologic signals, the signal trend indicating the temporal variation of the signal feature;
dynamically generating first and second transformations using at least one characteristic measure of the signal trend;
transforming a first portion of the signal trend into a first transformed signal trend using the first transformation, and transforming a second portion of the signal trend into a second transformed signal trend using the second transformation, the second portion of the signal trend different from the first portion of the signal trend; and
detecting a target physiologic event in response to the transformed signal trend meeting a specified criterion.

14. The method of claim 13, wherein transforming the first and second portions of the signal trends includes transforming the first portion of the signal trend non-overlapping in time with the second portion of the signal trend.

15. The method of claim 13, wherein:
the second portion of the signal trend includes data from the signal trend preceding the first portion of the signal trend in time, the second portion of the signal trend representing a baseline free of predicted target physiologic event; and
detecting a target physiologic event including determining whether a relative difference between the first and second transformed signal trends meets a specified criterion.

16. The method of claim 13, wherein dynamically generating the first and second transformation includes generating respectively first and second plurality of weight factors proportional to strength of the signal trend.

17. The method of claim 13, wherein dynamically generating the transformation includes generating respectively first and second plurality of timing-varying weight factors.

18. The method of claim 17, wherein generating the first and second plurality of time-varying weight factors includes determining values of the time-varying weight factors as one of a linear, a nonlinear, a monotonically increasing, or a monotonically decreasing function of relative time of the signal trend with respect to a reference time.

19. The method of claim 18, wherein generating the first and second plurality of time-varying weight factors includes determining the first plurality of time-varying weight factors as a monotonically increasing function of relative time of the first portion of the signal trend with respect to a first reference time, and determining the second plurality of time-varying weight factors as a monotonically decreasing function of relative time of the second portion of the signal trend with respect to a second reference time.

20. The method of claim 13, further comprising receiving an auxiliary signal non-identical to the one or more physiologic signals, wherein dynamically generating the transformation includes generating a plurality of weight factors proportional to strength of the auxiliary signal.
